(19) United States
(12) Patent Application Publication
(10) Pub. No.: US 2016/0375436 A1
(43) Pub. Date: Dec. 29, 2016

(54) DETECTION DEVICE

(71) Applicant: National Tsing Hua University, Hsinchu City (TW)

(72) Inventors: Chao-Min CHENG, Hsinchu City (TW); Mei-Jou TSAI, Hsinchu City (TW)

(21) Appl. No.: 14/868,122

(22) Filed: Sep. 28, 2015

(30) Foreign Application Priority Data
Jun. 26, 2015 (TW) 104120708

(51) Int. Cl. B01L 3/00; G01N 33/02

(52) U.S. Cl. CPC B01L 3/5023; G01N 33/02; B01L 2300/0825; B01L 2200/02; B01L 2300/089; B01L 2300/12; B01L 2300/165; B01L 2300/069

(57) ABSTRACT

A detection device includes a carrying unit, at least a reaction unit, a wire transporting unit, and a guiding unit. The carrying unit includes at least a hole, and the reaction unit is disposed on one side of the hole. The reaction unit includes at least a reaction area, which includes a hydrophilic material containing a detection reagent. The wire transporting unit is disposed on the other side of the hole and includes a sampling end and a transporting end. The guiding unit is in contact with the reaction unit and the transporting end. The guiding unit collects a test sample through the transporting end and provides at least a part of the test sample to react with the detection reagent. The detection device adopts the wire transporting unit to replace the pipette for collecting the test sample without directly contacting the detection area with the sample source.

### CROSS REFERENCE TO RELATED APPLICATIONS

This Non-provisional application claims priority under 35 U.S.C. §119(a) on Patent Application No(s). 104120708 filed in Taiwan, Republic of China on Jun. 26, 2015, the entire contents of which are hereby incorporated by reference.

### BACKGROUND OF THE INVENTION

Field of Invention

The present invention relates to a detection device and, in particular, to a detection device, which can transport the test sample to the reaction unit through a wire transporting unit based on capillary phenomenon.

Related Art

As the rising of health consciousness, the concept of self-detection in house is more and more popular. The self-detection allows the users to easily and simply detect the simple physiological status or the food safety anytime and anywhere. The detection of physiological status can help the general users to check the health statuses of themselves and, in particular, assist the patients to enhance the therapeutic effects and to control the diseases progressions. The food safety detections have become more and more important. This is because the food products may contain some remained bad substances, which exist in the raw materials or are generated during the manufacturing processes. Therefore, it is desired to effectively detect the food additives before tasting food products.

There are many detection methods for detecting food additives such as spectrophotometry, HPLC (high performance liquid chromatography), GC (gas chromatography), IC (ion chromatography), CE (capillary electrophoresis), polarography and FIA (flow injection analysis). However, these detection methods all need a high-tech and expensive equipment for detection, and require operators and a lot of time to precisely detect the additives in the labs.

Compared with the above laboratory detection methods, the self-detection has the advantages of convenience and low cost. The commonly used self-detection for detecting food additives mainly utilizes a testing strip containing a detection reagent to detect the food additives according to colorimetric method or photometric method. The user can compare the color of the testing strip with a given color table so as to determine the nitrite concentration in the food product. This simple measuring method brings the users an extremely large convenience and safety. However, the user has to carry the testing strip all the time, which may bother the users indeed. In addition, the existing testing strips are almost made by many processes, and the added substances in the testing strips will cause the risk of the safety of the testing strips.

Besides, it is considered to use a pipette to obtain the test sample so as to avoid the direct contact of the testing strip and the test sample. However, the pipette may retrieve over dose of the test sample so as to cause the waste of sample. Moreover, in order to obtain a precise testing result, the pipette must be well cleaned before the testing procedure, which makes the detection procedure more complex.

Therefore, it is an important subject to provide a detection device, which has the simple operation property as the existing testing strips and is advantageous in simplified preparing process before testing and an easy and fast operation, thereby improving the sampling and detecting speeds and the application safety.

### SUMMARY OF THE INVENTION

In view of the foregoing, an objective of the present invention is to provide a detection device, which has the simple operation property as the existing testing strips and is advantageous in simplified preparing process before testing and an easy and fast operation, thereby improving the sampling and detecting speeds and the application safety.

To achieve the above objective, the present invention discloses a detection device including a carrying unit, at least a reaction unit, a wire transporting unit, and a guiding unit. The carrying unit includes at least a hole, and the reaction unit is disposed on one side of the hole. The reaction unit includes at least a reaction area, which includes a hydrophilic material. The hydrophilic material contains a detection reagent. The wire transporting unit is disposed on the other side of the hole with respect to the reaction unit. The wire transporting unit includes a sampling end and a transporting end. The guiding unit is in contact with the reaction unit and the transporting end of the wire transporting unit. The guiding unit collects a test sample through the transporting end and provides at least a part of the test sample to react with the detection reagent.

In one embodiment, the detection device is cooperated with a tea bag.

In one embodiment, the wire transporting unit transports the test sample along a first direction, the guiding unit transports the test sample along a second direction, and an included angle is formed between the first direction and the second direction.

In one embodiment, the included angle is between 20 and 90 degrees.

In one embodiment, the carrying unit includes a plurality of holes, and the detection device further includes a plurality of reaction units disposed aside the holes, respectively.

In one embodiment, the reaction units are arranged along a straight line.

In one embodiment, the reaction units are arranged in a polygon shape or a concentric square shape.

In one embodiment, the reaction unit further includes a plurality of reaction areas arranged in a polygon shape or a concentric square shape.

In one embodiment, at least a part of the guiding unit is an extension structure of the transporting end, and the materials of the guiding unit and the wire transporting unit include cotton fiber.

In one embodiment, the guiding unit has a wire structure, a rod structure or a ball structure.

In one embodiment, the detection device further includes a covering unit disposed at the other side of the wire transporting unit with respect to the carrying unit. The covering unit and the carrying unit form a space for accommodating the wire transporting unit.

In one embodiment, the carrying unit has at least a non-reaction area, which is processed by a hydrophobic surface treatment.

In one embodiment, the non-reaction area surrounds the reaction area, and the hydrophilic material is exposed for absorbing the test sample collected by the sampling end.

As mentioned above, the detection device of the invention has a guiding unit connecting to the wire transporting unit and the reaction unit, so that the test sample adopted by the wire transporting unit can be transported to the reaction unit through the guiding unit for the following reaction and detection. The detection device of the invention utilizes the wire transporting unit as a bridge for absorbing the test sample and transporting it (by capillary phenomenon) to the reaction unit. Accordingly, the sampling procedure can be efficiently simplified.

Besides, the food products may not be served after being detected by the conventional testing strips, which need to directly contact the sample for detection. In contrary, the detection device of the invention has a wire transporting unit interrupted between the reaction unit and the test sample. In other words, the wire transporting unit can replace the pipette, so that the test sample can be transported to the reaction unit for detection without using the conventional pipette. In addition, the food products can still be served after being detected by the detection device of the invention.

### BRIEF DESCRIPTION OF THE DRAWINGS

The invention will become more fully understood from the detailed description and accompanying drawings, which are given for illustration only, and thus are not limitative of the present invention, and wherein:

FIG. 1A is a schematic diagram of a detection device according to a preferred embodiment of the invention;

FIG. 1B is a sectional diagram showing the detection device of FIG. 1A in operation;

FIG. 1C is a sectional view along the line A-A of FIG. 1A;

FIGS. 2 and 3 are sectional views of a detection device according to another preferred embodiment of the invention;

FIGS. 4 and 5 are sectional views of a detection device according to another preferred embodiment of the invention;

FIGS. 6 and 7 are top views of a detection device according to another preferred embodiment of the invention;

FIG. 8 is a sectional view of a detection device according to another preferred embodiment of the invention;

FIG. 9 is a sectional view of a detection device according to another preferred embodiment of the invention;

FIG. 10 is a schematic diagram of a detection device according to another preferred embodiment of the invention;

FIG. 11 is a schematic graph showing the mean intensities with respect to different reaction area layouts in the detection device 1i;

FIG. 12 is a schematic graph showing the absorption volumes of the test sample with respect to the wire transporting units 112i of the detection device 1i with different lengths;

FIG. 13 is a schematic diagram showing the detection results as using the detection device 1j to detect nitrite;

FIG. 14A is a schematic graph showing the mean intensity result as using the detection device 1j to detect BSA of different concentrations;

FIG. 14B is a schematic graph showing the color result as using the detection device 1j to detect BSA of different concentrations;

FIG. 15A is a schematic graph showing the mean intensity result as using the detection device 1j to detect uric acid of different concentrations;

FIG. 15B is a schematic graph showing the color result as using the detection device 1j to detect uric acid of different concentrations; and

FIG. 16 is a schematic graph showing the color result as using the detection device 1j to detect zinc of different concentrations.

### DETAILED DESCRIPTION OF THE INVENTION

The present invention will be apparent from the following detailed description, which proceeds with reference to the accompanying drawings, wherein the same references relate to the same elements.

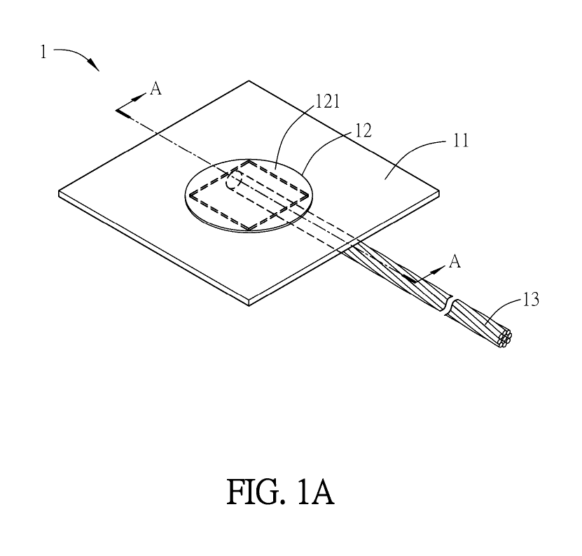

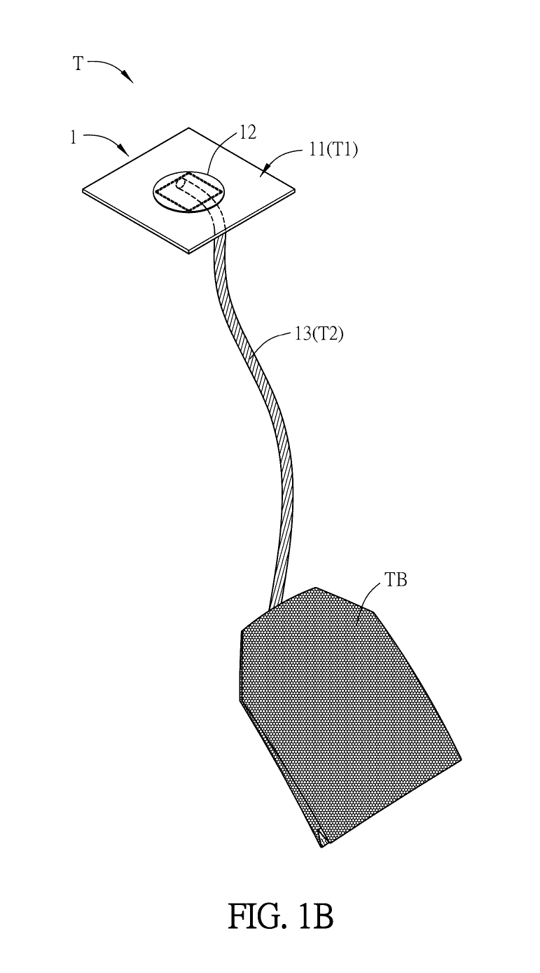

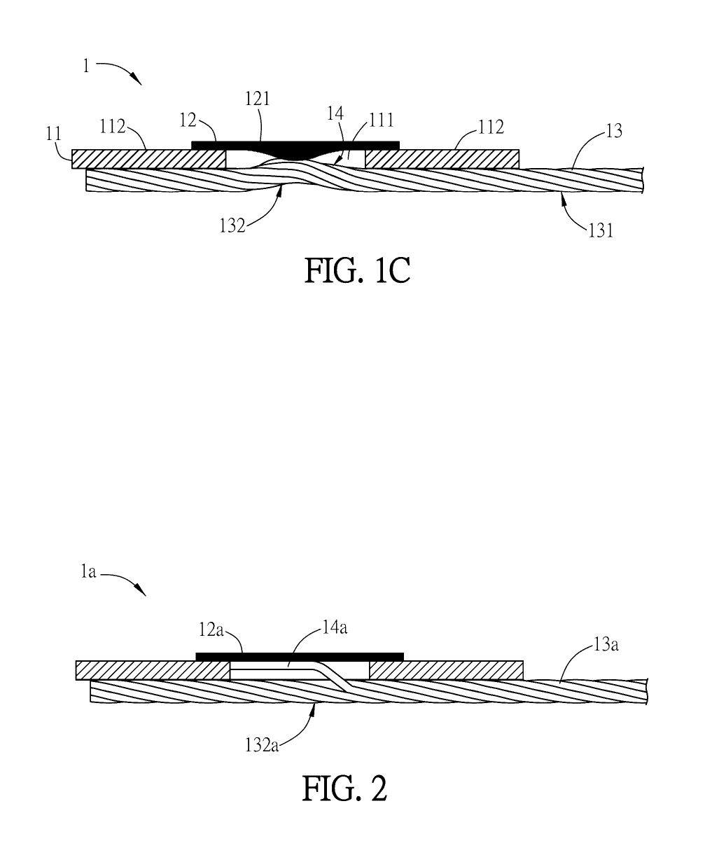

FIG. 1A is a schematic diagram of a detection device according to a preferred embodiment of the invention, FIG. 1B is a sectional diagram showing the detection device of FIG. 1A in operation, and FIG. 1C is a sectional view along the line A-A of FIG. 1A. Referring to FIGS. 1A to 1C, the detection device 1 of this embodiment is used to collect and detect a test sample, which is for example but not limited to a biological fluid or food product. Although this embodiment does not limit the type of the test sample, the following descriptions will focus on the food safety related samples such as the tea or tea drink made by tea bags.

In order to be properly applied to the daily necessities, the detection device 1 can be made as a part of a tea bag T (see FIG. 1B), such as the tag T1 and the cotton thread T2. Accordingly, it is possible to give an additional function (detection function) to the tea bag T. In other words, the tea bag T is a kind of essential products for many users, which means that the users do not carry any additional necessity along with themselves for the detection purpose. In this embodiment, the detection device 1 is cooperated with the tea bag body TB.

Referring to FIGS. 1A to 1C, the detection device 1 of the embodiment includes a carrying unit 11, a reaction unit 12 and a wire transporting unit 13. While viewing as a whole, the reaction unit 12, the carrying unit 11 and the wire transporting unit 13 are stacked in order from the top to the bottom. In more detailed, the carrying unit 11 includes at least a hole 111, which penetrates through two surfaces of the carrying unit 11. The reaction unit 12 is disposed on one side of the hole 111, while the wire transporting unit 13 is disposed on the opposite side of the hole 111. The wire transporting unit 13 is glued on the carrying unit 11. To be noted, the size and structure of the hole 111 are not limited, and any hole 111 can be matched to the reaction unit 12 and the wire transporting unit 13 is applicable.

Regarding to the functions, the carrying unit 11 is, for example, the tag T1 of the tea bag T for carrying the reaction unit 12, which is used in the detection procedure. The wire transporting unit 13 is, for example, the cotton thread T2 of the tea bag T. In this case, the wire transporting unit 13 has a sampling end 131 and a transporting end 132. The sampling end 131 is used to absorb the test sample and then transport it to the transporting end. In this embodiment, the wire transporting unit 13 is made of cotton fiber and, preferably, made by twisting a plurality of cotton fibers, so that it can have a good absorption ability. In practice, the material of the wire transporting unit 13 is not limited, and any material with absorption and hydrophilic properties is selectable.

To be noted, the sampling end 131 is defined as a part of the wire transporting unit 13 away from the carrying unit 11, and the transporting end 132 is defined as the residual part of the wire transporting unit 13. In other words, the sampling end 131 and the transporting end 132 are not structurally limited to the two end portions of the wire transporting unit 13.

In this embodiment, the reaction unit 12 has a reaction area 121, which includes a hydrophilic material containing a detection reagent. In this case, the detection reagent is selected according to the requirement of the detection. The method for disposing the detection reagent in the hydrophilic material is familiar to the skilled person in the art, so the detailed description thereof will be omitted. For example, the detection reagent (liquid) is disposed on the reaction area 121 and then the water therein is removed. The hydrophilic material contained in the reaction area 121 is also not limited. For example, the hydrophilic material can be a fiber substrate. The reaction area 121 can keep and absorb the test sample based on the capillary phenomenon generated by the fiber substrate (hydrophilic material). In this embodiment, the reaction area 121 and the reaction unit 12 are substantially equivalent areas.

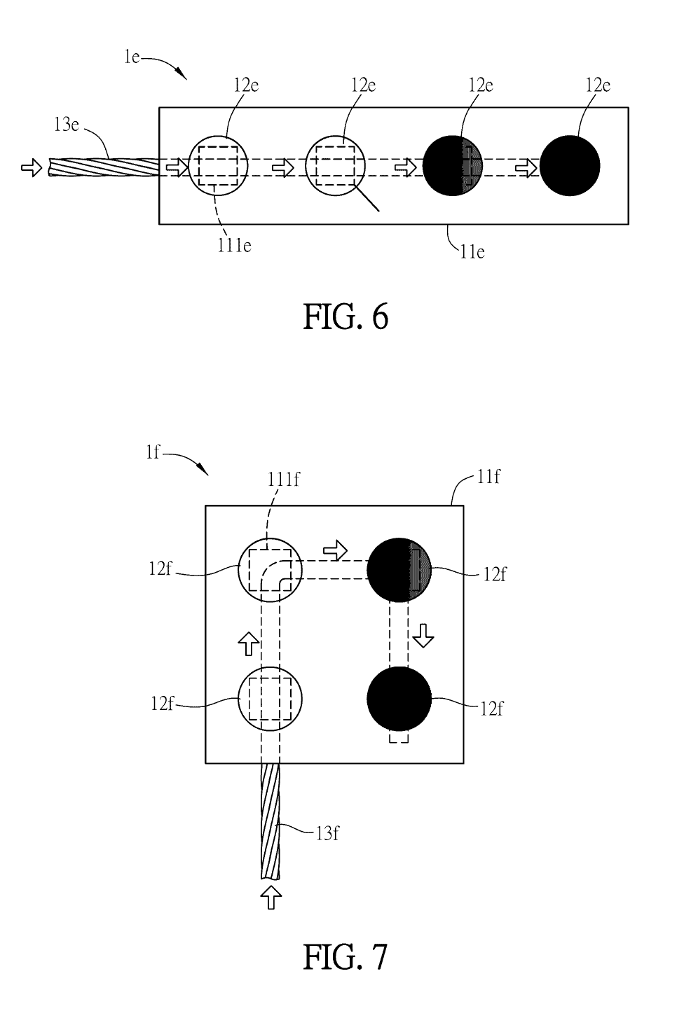

To be noted, the number of the reaction units is not limited. In some embodiments, as shown in FIG. 6, the detection device 1e includes a plurality of reaction units 12e and a plurality of holes 111e corresponding to the reaction units 12e. The design of the detection device is variable and depended on the requirement of detection.

Besides, in order to prevent the detection reagent from exposing in the air too long, which may affect the lifetime of the detection reagent, the detection device of another embodiment further includes a gel to cover the detection reagent so as to generate a mixed slurry. This mixed slurry is a costate of the gel and the detection reagent. In some cases, the mixed slurry also includes the solidified mixture of the gel and the detection reagent. Since the detection reagent is covered by the gel, it is possible to decrease the contact area between the detection reagent and air so as to effectively extend the lifetime of the detection reagent. Herein, the above-mentioned gel is a polymer material such as PVA (polyvinyl alcohol).

The mixed slurry can be prepared by the following steps. At first, a liquid gel and a liquid detection reagent are provided. In this embodiment, the liquid gel is a viscous liquid composed of PVA (mol. wt. 70,000˜100,000, 10-15%) and water, wherein the viscosity thereof is about 8,000˜20,000 CPS. To be noted, the viscosity of PVA is not limited to the above example, and the viscosity of the mixed slurry can be properly controlled by adjusting the ratio of PVA and water.

The structures of the reaction unit 12 and the carrying unit 11 will be further described hereinafter. With reference to FIGS. 1A and 1C, the carrying unit 11 has a plate structure covered by a hydrophobic material, so that it can be defined as a hydrophobic area. When the reaction unit 12 and the carrying unit 11 are assembled, the carrying unit 11 (the hydrophobic area) surrounds the reaction unit 12 (reaction area 121). In other words, the carrying unit 11 has at least one non-reaction area 112 for isolating the reaction area 121. In this embodiment, the non-reaction area 112 is formed by wax printing. The general wax printing method for forming the hydrophobic non-reaction area 112 is to utilize a proper apparatus with wax ejecting function (e.g. a printer) to print the wax in a preset pattern, shape or size on the carrying unit 11. Accordingly, the configuration of the non-reaction area 112 can effectively restrict the test sample in the reaction area 121, thereby preventing the loss of the sample and improving the detection accuracy.

The method for forming the non-reaction area 112 is not limited to the above-mentioned method. In practice, it is also possible to form the non-reaction area 112 by coating a photoresist layer on the carrying unit 11. For example, when coating a SU-8 epoxy-based negative photoresist on the carrying unit 11, the part of the photoresist irradiated by UV light will not be removed by the developing solution. Thus, the remained photoresist can form the hydrophobic non-reaction area 112. This and other similar methods for forming the non-reaction area 112 are known by the skilled person in the art, so the detailed descriptions thereof will be omitted.

When the user puts the tea bag body TB in the liquid test sample, the tea bag body TB can absorb the liquid test sample and transport it to the cotton thread T2 (the wire transporting unit 13). Of course, in practice, the sampling end 131 of the wire transporting unit 13 can directly contact and collect the test sample. In order to transport the test sample from the transporting end 132 of the wire transporting unit 13 to the reaction unit 12, the detection device 1 further includes a guiding unit 14, which is located in the hole 111 of the carrying unit 11. The guiding unit 14 connects the reaction area 121 of the reaction unit 12 and the transporting end 132 of the wire transporting unit 13. In other words, the guiding unit 14 is an intermedia in the hole 111 for communicating the reaction area 121 and the transporting end 132. To achieve the above purpose, the guiding unit 14 is also selected from the materials with absorbing and hydrophilic abilities. For example, the guiding unit 14 and the wire transporting unit 13 of the embodiment are all made of cotton fiber material. Referring to FIG. 1C, the guiding unit 14 can be composed of a part of the reaction unit 12 and a part of the wire transporting unit 13. In practice, a part of the reaction unit 12 protrudes into the hole 111, and a part of the wire transporting unit 13 also protrudes into the hole 111. The protruding parts of the reaction unit 12 and the wire transporting unit 13 are connected to form the guiding unit 14.

The method for transporting the test sample by the detection device 1 of the embodiment will be further described hereinafter. With reference to FIG. 1C, the test sample is transported through the wire transporting unit 13 along a first direction D1, and then transported through the guiding unit 14 to the reaction unit 12 along a second direction D2. Herein, the first direction D1 and the second direction D2 have an included angle, which is between 20 and 90 degrees. Accordingly, the guiding unit 14 can assist to deliver the test sample and change the transporting direction of the test sample in the hole 111.

In other embodiments, the wire transporting unit and the reaction unit can be made of different materials, preferably with different absorption abilities. For example, the absorption ability (for the test sample) of the reaction unit is better than that of the wire transporting unit, so that the reaction unit and the wire transporting unit have the competition effect to the test sample. Accordingly, this competition effect can speed the delivery of the test sample from the wire transporting unit to the reaction unit, and control the test sample to move along a single direction so as to prevent the undesired countercurrent flow.

To be noted, the detection device 1 can provide multiple moving directions for the test sample. Thus, in view of the entire flowing procedure of the test sample, the detection device 1 is substantially a 3D detection device.

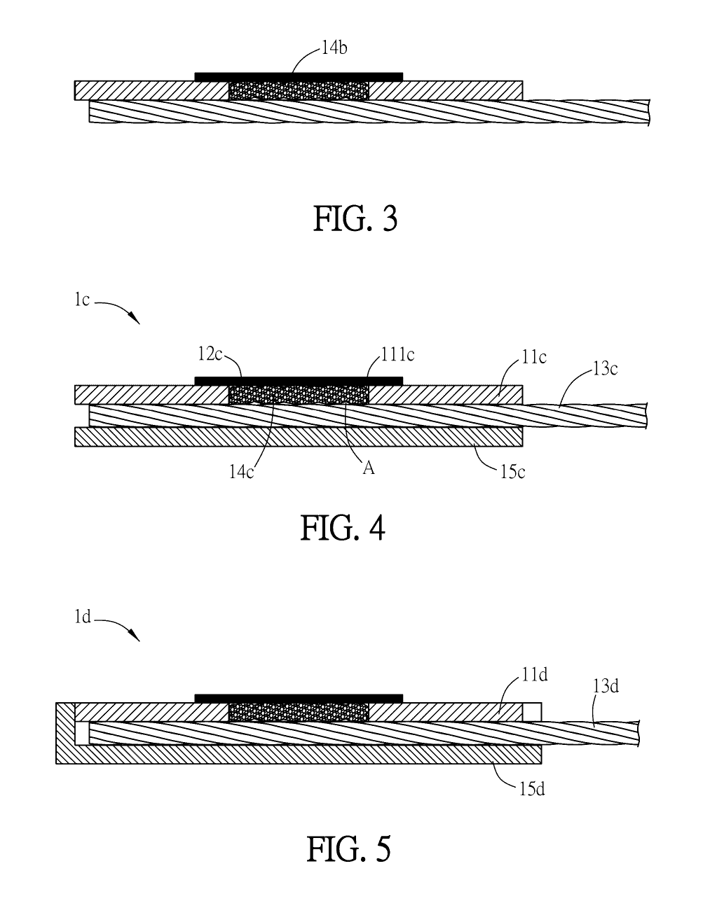

FIGS. 2 and 3 are sectional views of a detection device according to another preferred embodiment of the invention. Referring to FIG. 2, the structure and components of the detection device 1a of this embodiment are mostly the same as those of the previous embodiment, so the following description will focus on the undisclosed part of this embodiment. The guiding unit 14a of the detection device 1a is an extension structure of the transporting end 132a of the wire transporting unit 13a. That is, the guiding unit 14a is one of the multiple cotton fibers, which form the wire transporting unit 13a, and is a wire structure. The guiding unit 14a must contact with the reaction unit 12a for building up the entire transporting structure. To be noted, the number and arrangement of the cotton fibers in the guiding unit 14a are not limited in this embodiment. Any configuration that can properly transport the content of the test sample to the reaction unit 12a depending on the transporting requirement and the components in the liquid is acceptable.

To be noted, the aspects of the guiding unit are not limited to the above disclosed guiding units 14 and 14a. In other embodiments, the guiding unit 14b is an additional added component (see FIG. 3), and it is preferably made by twisting the cotton fibers into a ball structure or made into any shape of structure (e.g. a cylindrical structure) containing a hydrophilic material. Similarly, the arrangement of the guiding unit 14b is not limited in this embodiment, and any configuration that can simultaneously contact the reaction unit and the wire transporting unit for properly transporting the content of the test sample to the reaction unit depending on the transporting requirement and the components in the liquid is acceptable.

FIGS. 4 and 5 are sectional views of a detection device according to another preferred embodiment of the invention. The structure and components of the detection device 1c of this embodiment are mostly the same as those of the previous embodiment, so the following description will focus on the undisclosed part of this embodiment. The detection device 1c of this embodiment further includes a covering unit 15c disposed at the other side of the wire transporting unit 13c with respect to the carrying unit 11c. The parts of the covering unit 15c and the carrying unit 11c, which are protruded from the wire transporting unit 13c, are bonded (e.g. by adhering) so as to form a space for accommodating and fixing the wire transporting unit 13c. The configuration of the covering unit 15c can effectively align the wire transporting unit 13c and the hole 111c so as to assist the test sample to be transported to the reaction unit 12c at shortest time. Moreover, the configuration of the covering unit 15c can prevent the exposure of the hole 111c and the bottom surface of a part of the reaction unit 12c, thereby avoiding the undesired contamination of user's hands by the detection reagent and effectively improving the detection safety.

To be noted, when the detection device 1c containing the covering unit 15c is cooperated with the guiding unit 14c with 3D structure (e.g. a ball or rod shape), the guiding unit 14c can further create a supporting space A for accommodating the wire transporting unit 13c and the guiding unit 14c, thereby improving the flowing of the test sample and the detection accuracy.

To be noted, the aspects of the covering unit are not limited to the above disclosed covering units 15c. In other embodiments, as shown in FIG. 5, the covering unit 15d of the detection device 1d covers the wire transporting unit 13d and the surface of at least a part of the carrying unit 11d, which is close to the wire transporting unit 13d. This configuration can protect the detection procedure of the detection device 1d from external materials and objects, and thus improve the reliability of the test result.

FIGS. 6 and 7 are top views of a detection device according to another preferred embodiment of the invention. With reference to FIGS. 6 and 7, the structure and components of the detection devices 1e and 1f of this embodiment are mostly the same as those of the previous embodiment, so the following description will focus on the undisclosed part of this embodiment. The detection devices 1e and 1f of this embodiment include a plurality of reaction units 12e and 12f, which are disposed corresponding to the holes 111e of the carrying unit 11e and the holes 111f of the carrying unit 11f. The reaction units 12e and 12f can be arranged in a straight line (see FIG. 6) or a polygon shape (see FIG. 7). Of course, they can also be arranged in other shapes (e.g. a concentric square shape) depending on the detection requirement. In this case, the wire transporting units 13e and 13f are disposed corresponding to the locations of the reaction units 12e and 12f. As a result, the detection devices 1e and 1f having a plurality of reaction units 12e and 12f can be utilized to multiple detection targets, thereby providing the multiple detection function and increasing the utility of the detection devices 1e and 1f.

To be noted, the structure of the detection devices 1e and 1f of this embodiment is not limited to the 3D structure. The detailed structure and components of the detection devices 1e and 1f can be referred to the designs of the detection devices as shown in FIGS. 1A to 5. Accordingly, it is possible to flexibly design the proper detection device structure according to the actual requirement.

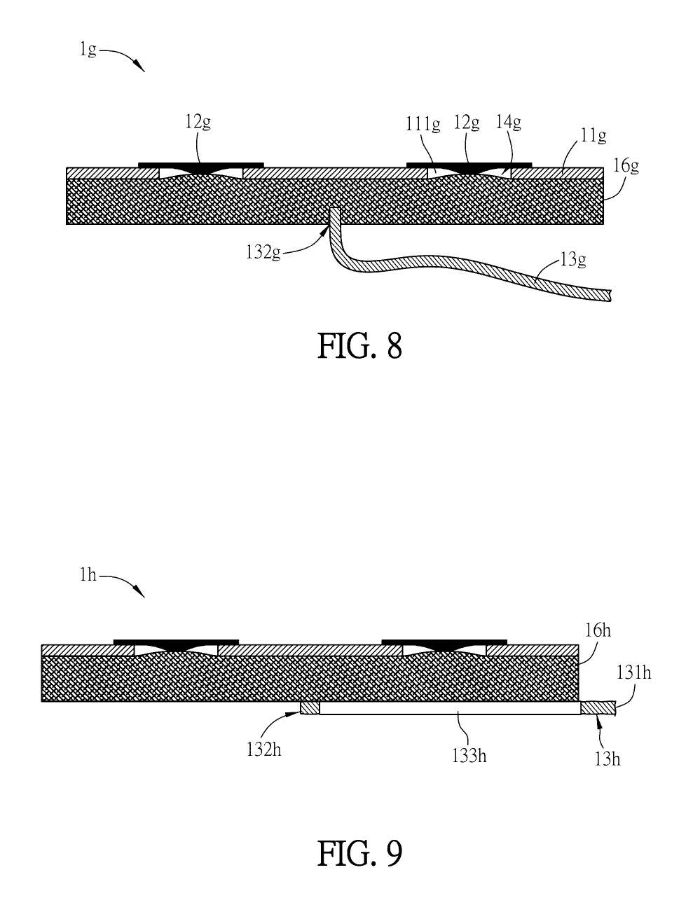

FIG. 8 is a sectional view of a detection device according to another preferred embodiment of the invention. With reference to FIG. 8, the structure and components of the detection device 1g of this embodiment are mostly the same as those of the previous embodiment except for that a hydrophilic structure 16g is disposed between the carrying unit 11g and the wire transporting unit 13g. The shape of the hydrophilic structure 16g is almost the same as that of the carrying unit 11g, but the thickness of the hydrophilic structure 16g is substantially greater than that of the carrying unit 11g. Besides, the material of the hydrophilic structure 16g is not limited and can be selected from any material capable of absorbing the liquid test sample. For example, this embodiment utilizes cotton as the material of the hydrophilic structure 16g. In the transporting route of the test sample, the hydrophilic structure 16g is functioned as an extension of the transporting end 132g of the wire transporting unit 13g. The hydrophilic structure 16g contacts the a part of the reaction unit 12g in the hole 111g so as to form the guiding unit 14g. According to the structure of the detection device 1g of this embodiment, the test sample can be transported to the transporting end 132g (and the hydrophilic structure 16g) through the sampling end 131g of the wire transporting unit 13g. Furthermore, the test sample can be transported to each reaction unit 12g according to the absorption of the hydrophilic structure 16g. Preferably, the wire transporting unit 13g is connected to the hydrophilic structure 16g by only the transporting end 132g and thus disposed at the center position of the hydrophilic structure 16g. In more specific, the wire transporting unit 13g is disposed at the center position of the bottom surface of the detection device 1g. When the test sample is transported to the hydrophilic structure 16g, it can be diffused from this center position. This configuration can prevent the dilution problem after traveling for a long distance, thereby effectively improving the detection accuracy.

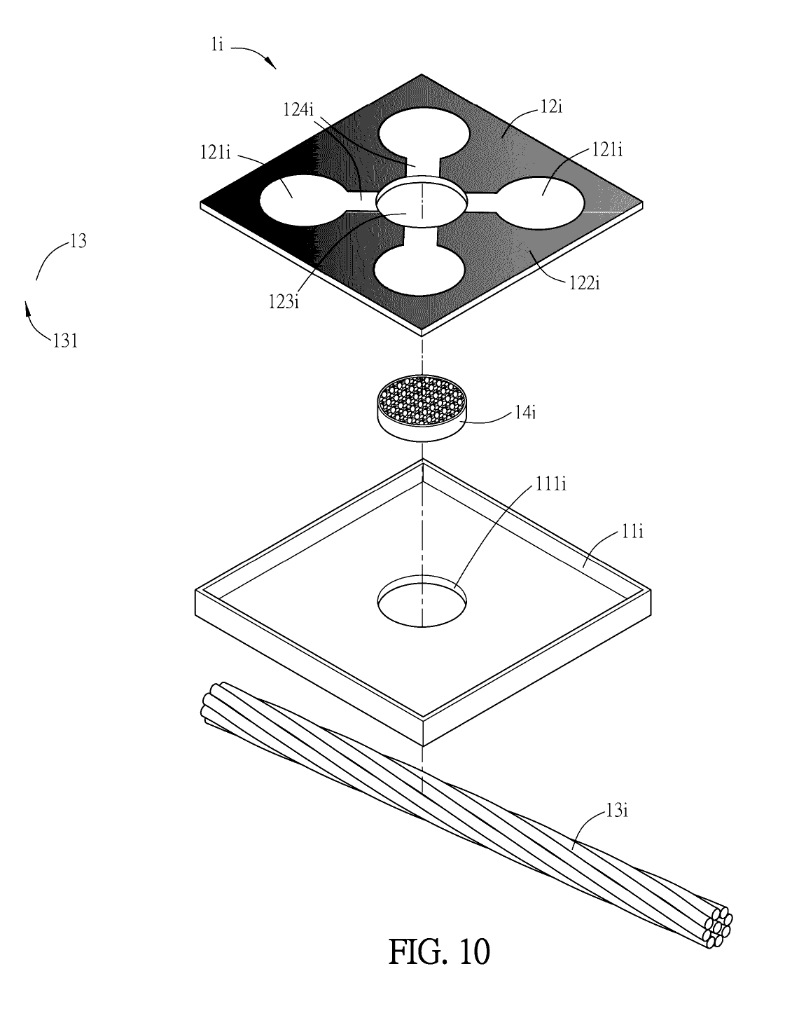

The structure of the detection device 1g of FIG. 8 can be modified to the configuration as shown in FIG. 9. FIG. 9 is a sectional view of a detection device according to another preferred embodiment of the invention. In this embodiment, the structure and components of the detection device 1h of this embodiment are mostly the same as those of the detection device 1g of the previous embodiment. The different is that most structure of the wire transporting unit 13h of the detection device 1h is attached to the hydrophilic structure 16h except for the transporting end 132h. In other words, the total surface of the wire transporting unit 13h attached to the hydrophilic structure 16g is increased, thereby enhancing the connection between the wire transporting unit 13h and the hydrophilic structure 16g.

To be noted, the detection device 1h of this embodiment can also prevent the dilution problem after test sample travels for a long distance. In this embodiment, the wire transporting unit 13h further has an isolation member 133h, which covers a part of the wire transporting unit 13h. In more specific, the isolation member 133h covers the part of the wire transporting unit 13h attached to the hydrophilic structure 16h except for the transporting end 132h, and the sampling end 131h of the wire transporting unit 13h is exposed from the detection device 1h for contacting and collecting the test sample.

According to the above structure design, the detection device 1h can enhance the connection between the wire transporting unit 13h and the hydrophilic structure 16h. When the wire transporting unit 13h (the transporting end 132h) and the hydrophilic structure can form a smooth flow channel for transporting the test sample, thereby preventing the dilution problem after the test sample travels for a long distance and thus improving the detection accuracy.

FIG. 10 is a schematic diagram of a detection device according to another preferred embodiment of the invention. Referring to FIG. 10, the detection device 1i also includes a carrying unit 11i, a reaction unit 12i, a wire transporting unit 13i and a guiding unit 14i. The structure and components of the detection device 1i are mostly the same as those of the previous embodiment. The guiding unit 14i is disposed in the hole 111i of the carrying unit 11i. The reaction unit 12i includes a plurality of reaction areas 121i and a non-reaction area 122i surrounding the reaction areas 121i. The detection reagent is disposed in each of the reaction areas 121i. The numbers and shapes of the reaction areas 121i and the non-reaction area 122i are not limited to the embodiment, and they can be configured according to the actual detection requirement.

The structure of the reaction unit 12i will be further described hereinafter. In this embodiment, the reaction unit 12i is composed of hydrophobic fibers, and a part of the hydrophobic fibers is treated by O₂ Plasma, argon Plasma or air Plasma so as to form and define the desired hydrophilic fibers. The formed hydrophilic fibers defines the hydrophilic reaction area(s) 121i, while the residual hydrophobic fibers defines the non-reaction area 122i. To be noted, the method for forming the reaction unit 12i is not limited to the above aspect. In practice, the reaction unit 12i can also be composed of hydrophilic fibers, and a part of the hydrophilic fibers is treated by wax printing and baking (100° C., 10 minutes) so as to form the pattern of the hydrophobic non-reaction area 122i. The residual hydrophilic fibers out of the wax printing pattern form the reaction areas 121i. In this embodiment, the reaction unit 12i includes a hole 123i for accommodating a part of the guiding unit 14i, and the reaction areas 121 are connected to the guiding unit 14i in the hole 123i through a plurality of hydrophilic channels 124i, which can absorb and transport the test sample from the reaction unit 12i.

In this embodiment, the reaction areas 121i are arranged in a polygon shape. Of course, they can also be arranged in other shapes (e.g. a concentric square shape) based on the actual detection requirement.

According to the above structure, the test sample located in each single area (the hole 123i and the guiding unit 14i) can be transported to each reaction area 121i via the channel 124i. Therefore, the traveling distance of the test sample in the reaction unit 12i can be effectively minimized, thereby preventing the dilution problem after the test sample travels for a long distance and thus effectively improving the detection accuracy.

To be noted, the detection devices of the above embodiments may further include a covering structure (not shown) covering the reaction areas. The covering structure is, for example but not limited to, an adhesion, and the state of the adhesion is, not limited to, liquid, gel or solid. Any aspect of the adhesion that can properly isolate the reaction area is acceptable. In this case, the covering structure can cover the reaction area and the detection reagent, so that it is possible to reduce the contact area between the detection reagent and air so as to extend the lifetime thereof.

Moreover, the material of the covering structure can be selected from a polymer material such as, for example but not limited to, PVA (polyvinyl alcohol). This example is not to limit the embodiment. In practice, other transparent water-soluble materials can be used to form the covering structure.

The above-mentioned detection devices can be applied to test any suitable test sample and not limited. When the detection device of the invention is used in bio detection and the test sample is blood, the detection reagent may include a glucose detection reagent or a urea nitrogen detection reagent. When the test sample is saliva, the detection reagent may include a pH detection reagent, a glucose detection reagent, a uric acid detection reagent or a nitrite detection reagent. When the test sample is urine, the detection reagent may include a glucose detection reagent, a nitrite detection reagent, a pH detection reagent, a urinary protein, a bilin detection reagent, a bilirubin detection reagent or a ketone detection reagent. When the test sample is tear, the detection reagent may include a glucose detection reagent. When the test sample is vaginal discharges, the detection reagent may include a pH detection reagent, a glycogen detection reagent or a lactate detection reagent. When the test sample is tissue fluid of skin wound, the detection reagent may include antigen of type XVII collagen with NC16A domain, anti-IgG antibody conjugated with HRP, 3,3′,5,5′-tetramethylbenzidine and dihydrogen dioxide.

The glucose detection reagent includes 75 U/mL glucose oxidase, 15 U/mL horseradish peroxidase and 0.6M potassium iodide. The urea nitrogen detection reagent includes 5% (w/v) p-dimethylaminobenzaldehyde. The pH detection reagent includes bromothymol blue and resazurin. The uric acid detection reagent includes 2.56% (w/v) 2,2′-biquinoline-4,4′-dicarboxylic acid disodium salt hydrate, 20 mM sodium citrate, and 0.08% (w/v) copper(II) sulfate. The nitrite detection reagent includes 50 mM sulfanilamide, 330 mM citric acid, and 10 mM N-(1-naphthyl) ethylenediamine dihydrochloride. The protein detection reagent includes 250 mM citric acid and 3.9 mM tetrabromophenol blue. The blood detection reagent includes 3% hydrogen peroxide and 3,3′,5,5′-tetramethylbenzidine. The bilin detection reagent includes 0.1M p-dimethylaminobenzaldehyde and 0.1M hydrogen chloride. The bilirubin detection reagent includes 4.9 mM sodium nitrite, 145 mM sulfanilic acid and 104 mM hydrogen chloride. The ketone detection reagent includes 3% sodium pentacyanonitrosylferrate(III) dihydrateide and 0.2M glycine. The glycogen detection reagent includes 2 U/mL glucose oxidase, 2 U/mL horseradish peroxidase and Oxired. The lactate detection reagent includes 2.8 U/mL lactate oxidase, 3.1 U/mL horseradish peroxidase and 3,3′,5,5′-tetramethylbenzidine. The above mentioned detection reagents can be used in the detection device of any above embodiment simultaneously or separately.

In another embodiment of the invention, when the detection device is used to detect food products, the detection reagent includes a nitrite detection reagent, a pH detection reagent, a BSA (bovine serum albumin) detection reagent, a carbamate detection reagent, an organophosphorus detection reagent, a paraquat detection reagent, a starch detection reagent, a cholesterol detection reagent, a tea polyphenol detection reagent, a glycogen detection reagent, a bilirubin detection reagent or a lactate detection reagent. The above mentioned detection reagents can be used in the detection device of any above embodiment simultaneously or separately.

In practice, the cholesterol detection reagent is to use a HRP system to detect the hydrogen peroxide generated from the reaction of cholesterol and cholesterol oxidase. In more detailed, in the detection of cholesterol, the cholesterol existing in the test sample can react with the cholesterol oxidase so as to generate hydrogen peroxide, and the generated hydrogen peroxide is reacted with 4-aminoantipyrine and peroxidase (POD) to generate red color (originally colorless). To detect the red color can confirm the existing of cholesterol.

Besides, when the detection device is used to detect food products, it can also detect the contained heavy metal such as, for example but not limited to, arsenic, lead, zinc and mercury.

The actual operation and effect of the detection device (e.g. the detection device 1i) will be discussed in the following experimental examples. To be noted, the following examples are for illustrations only so that the skilled person can realize and repeat this invention. Of course, the detection devices of other embodiments can also be used to achieve the same goal, and this invention is not limited.

### Experimental Example 1

### The Layout of the Reaction Areas and the Color Intensity Test

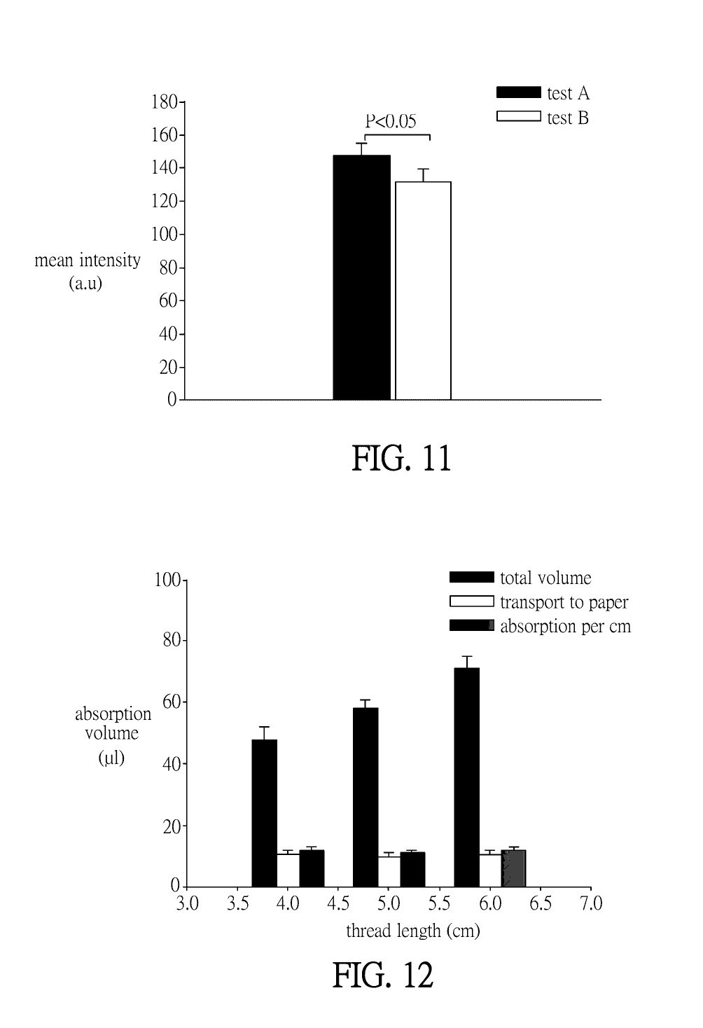

FIG. 11 is a schematic graph showing the mean intensities with respect to different reaction area layouts in the detection device 1i. Referring to FIG. 11, the example is to check whether the reaction unit 12i is configured with the hydrophobic non-reaction area 122i. A blue aqueous solution is transported through the wire transporting unit 13i to the detection device 1i (test A) and to a detection device without non-reaction area (test B). The color results of tests A and B are collected by naked eyes, camera or scanner. The experimental results indicate that the color intensities between the test A (using the detection device 1i with hydrophobic non-reaction area 122i) and the test B (using the detection device without hydrophobic non-reaction area) are obviously different.

### Experimental Example 2

### The Test for the Relationship Between the Length of the Wire Transporting Unit and the Absorption Volume

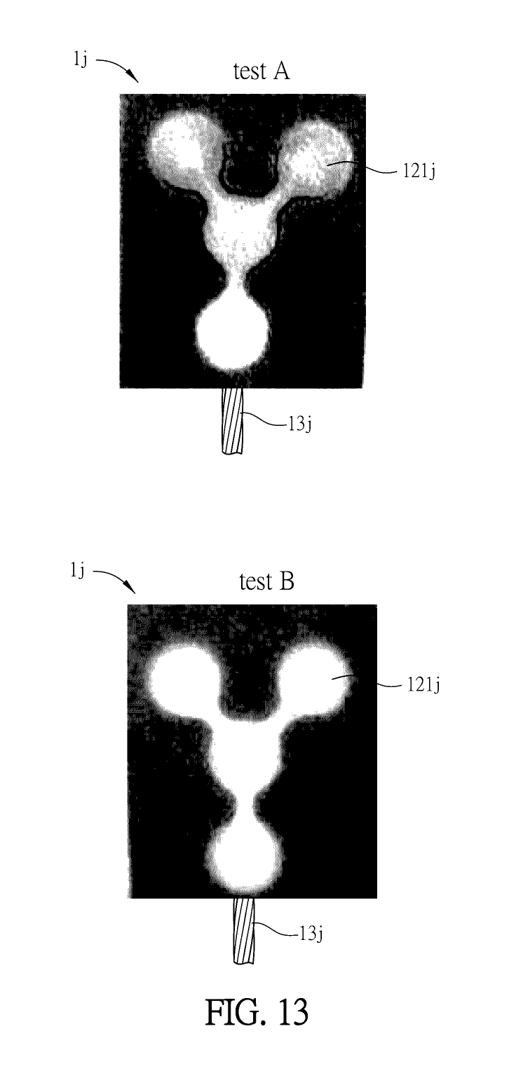

At first, a fine balance (sensitivity=1 mg) is used to measure the weight of a wire transporting unit 112i before absorbing water. Next, the wire transporting unit 112i is hanged on the edge of a beaker, and only the tail part (1-2 cm) of the wire transporting unit 112i is contact with solution. The solution in the beaker is absorbed by the wire transporting unit 112i based on capillary phenomenon until the entire wire transporting unit 112i (the other end hanged on the beaker) is also full filled with the solution. Then, the wire transporting unit 112i is weighted again. Afterwards, the wire transporting unit 112i is put on the reaction unit 12i (as the configuration of detection device 10. Accordingly, the reaction area 121i of the reaction unit 12i, which has better absorption ability, can absorb the solution from the wire transporting unit 112i. Then, the wire transporting unit 112i is weighted again after the reaction area 121i is filled with solution. This experiment can be done within 3 minutes, so the evaporation effect can be ignored. All experiments are performed under room temperature, so the density of water is 1 g/cm³ or 1000 mg/mL. That is, transporting 1 mg of water means to transport 1 μL of water. The experimental results are shown in FIG. 12.

FIG. 12 is a schematic graph showing the absorption volumes of the test sample with respect to the wire transporting units 112i of the detection device 1i with different lengths. In this embodiment, the wire transporting unit 112i replaces the conventional pipette for transporting the test sample to the reaction area 121i. With reference to FIG. 12, the absorption volume of the test sample and the length of the wire transporting unit are in positive proportion, which means that each centimeter of the wire transporting unit 112i can absorb the equivalent volume of test sample. According to the experimental result, the saturated volume of the reaction area 121i is not affected by the total length of the wire transporting unit 112i, so the reaction area 121i can absorb a constant amount of test sample in the saturated state. This feature can provide a stable reproducibility.

### Experimental Example 3

### Detecting Nitrite by the Detection Device 1j

To be noted, the structure of the applied detection device 1j is mostly the same as that of the detection device 1i of the previous embodiment. The difference therebetween is that the detection device 1j includes three reaction areas 121j.

The detection reagent is dropped by micropipette (Gilson, Inc.) onto the reaction areas 121j of the detection device 1j. The detection reagent includes 50 mmol/L sulfanilamide (≧99%, Sigma-Aldrich), 330 mmol/L citric acid (≧99.5%, Sigma-Aldrich) and 10 mmol/L N-(1-naphthyl)ethylene diamine (≧98%, Sigma-Aldrich). After adding the detection reagent, the detection device 1j is dried for 15 minutes at 25° C. Then, the wire transporting unit 13j of the detection device 1j is used to contact and collect the test samples. The test samples include a food sample containing nitrite standard diluted in hot pot soup (test A) and a buffer sample containing nitrite standard diluted in distilled deionized water (test B). Waiting for 7 minutes, the color intensity of the reaction area 121j is determined by ImageJ analysis software.

The analysis result is shown in FIG. 13. After the test sample reacts with the detection reagent, the test result indicates that the test A has obviously color intensity in the reaction area, but the test B has no color change in the reaction area.

### Experimental Example 4

### Detecting BSA (Different Concentrations) by the Detection Device 1j

The detection reagent is dropped by micropipette (Gilson, Inc.) onto the reaction areas 121j of the detection device 1j. The detection reagent includes 3.9 mM TBPB/95% ethanol and 250 mM citric acid (≧99.5%, Sigma-Aldrich). After adding the detection reagent, the detection device 1j is dried for 15 minutes at 25° C. Then, the wire transporting unit 13j of the detection device 1j is used to contact and collect the test samples. The test samples include BSA of different concentrations (0 μM, 1.875 μM, 3.75 μM, 7.5 μM, 15 μM and 30 μM). Waiting for 7 minutes, the color intensity of the reaction area 121j is determined by ImageJ analysis software.

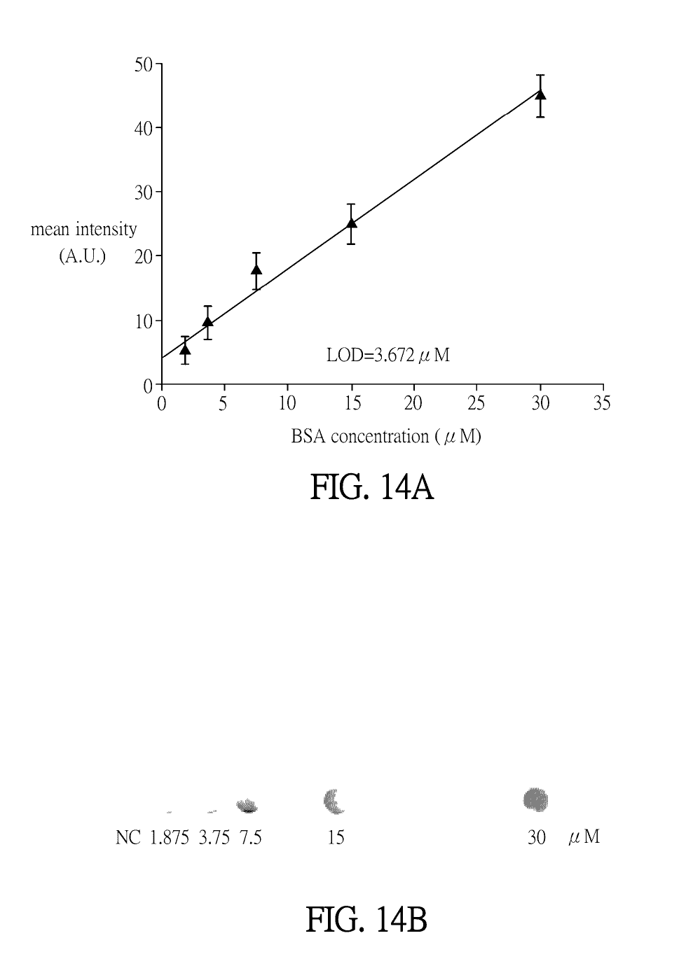

The analysis result is shown in FIGS. 14A and 14B. After the test samples reacts with the detection reagent, the test result indicates that the detected mean intensity of the color reaction increases as BSA concentration increases (see FIG. 14A). Comparing to the results of FIG. 14B, the color of the reaction areas 121j becomes darker as BSA concentration increases.

### Experimental Example 5

### Detecting Uric Acid (Different Concentrations) by the Detection Device 1j

The detection reagent is dropped by micropipette (Gilson, Inc.) onto the reaction areas 121j of the detection device 1j. The detection reagent includes 2.56% (w/v) 2.56% (w/v) 2,2′-biquinoline-4,4′-dicarboxylic acid disodium salt hydrate, 20 mM sodium citrate, and 0.08% (w/v) copper(II) sulfate. After adding the detection reagent, the detection device 1j is dried for 15 minutes at 25° C. Then, the wire transporting unit 13j of the detection device 1j is used to contact and collect the test samples. The test samples include uric acid of different concentrations (0 μM, 100 μM, 200 μM, 400 μM, 800 μM and 1600 μM). Waiting for 7 minutes, the color intensity of the reaction area 121j is determined by ImageJ analysis software.

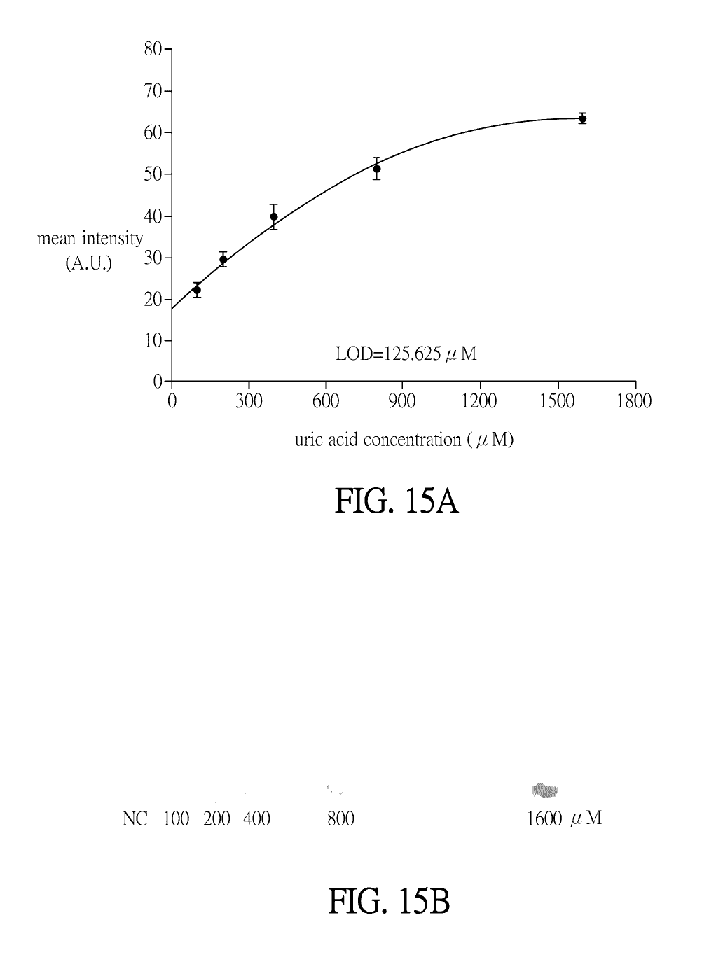

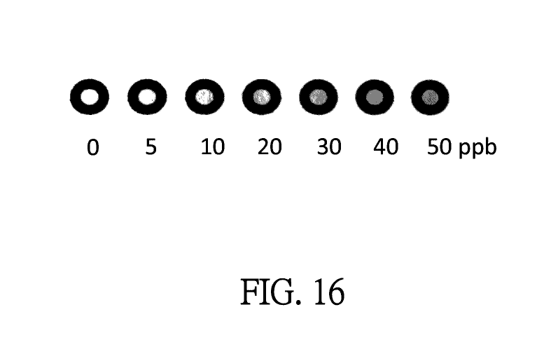

The analysis result is shown in FIGS. 15A and 15B. After the test samples reacts with the detection reagent, the test result indicates that the detected mean intensity of the color reaction increases as uric acid concentration increases (see FIG. 15A). Comparing to the results of FIG. 15B, the color of the reaction areas 121j becomes darker as uric acid concentration increases.

### Experimental Example 6

### Detecting Zinc (Zn²⁺) of Different Concentrations by the Detection Device 1

The detection reagent is dropped by micropipette (Gilson, Inc.) onto the reaction areas 121 of the detection device 1. The detection reagent includes a chelation (PAN). After adding the detection reagent, the detection device 1 is dried for 15 minutes at 25° C. Then, the wire transporting unit 13 of the detection device 1 is used to contact and collect the test samples. The test samples include zinc of different concentrations (0 ppb, 5 ppb, 10 ppb, 20 ppb, 30 ppb, 40 ppb and 50 ppb). Waiting for 7 minutes, the color intensity of the reaction area 121 is determined by ImageJ analysis software. To be noted, the detection device 1 can be placed in the oven (60° C.) so as to speed the evaporation of the solvent in the reaction areas.

The analysis result is shown in FIG. 16. After the test samples reacts with the detection reagent, the test result indicates that the color of the reaction areas 121 becomes darker as zinc concentration increases.

To be noted, the detection device of the embodiment can be applied to other heavy metals. In practice, the detection device also be used to detect other heavy metals such as mercury (Hg²⁺) or lead (Pb²⁺). The detection reagent for detecting mercury includes dithizone (0.5 mM) in acetone/NH₄Cl (5%/95%, v/v) (0.5 M of NH₄Cl with pH of 9.0). The detection reagent for detecting lead includes rhodizonic acid (0.01 mM) in tartaric acid (0.1 M, pH=2.9).

In summary, the detection device of the invention has a guiding unit connecting to the wire transporting unit and the reaction unit, so that the test sample adopted by the wire transporting unit can be transported to the reaction unit through the guiding unit for the following reaction and detection. The detection device of the invention utilizes the wire transporting unit as a bridge for absorbing the test sample and transporting it (by capillary phenomenon) to the reaction unit. Accordingly, the sampling procedure can be efficiently simplified.

Besides, the food products may not be served after being detected by the conventional testing strips, which need to directly contact the sample for detection. In contrary, the detection device of the invention has a wire transporting unit interrupted between the reaction unit and the test sample. In other words, the wire transporting unit can replace the pipette, so that the test sample can be transported to the reaction unit for detection without using the conventional pipette. In addition, the food products can still be served after being detected by the detection device of the invention.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A detection device, comprising:
a carrying unit comprising at least a hole;
at least a reaction unit disposed on one side of the hole, wherein the reaction unit comprises at least a reaction area, the reaction area comprises a hydrophilic material, and the hydrophilic material contains a detection reagent;
a wire transporting unit disposed on the other side of the hole with respect to the reaction unit, wherein the wire transporting unit comprises a sampling end and a transporting end; and
a guiding unit being in contact with the reaction unit and the transporting end of the wire transporting unit;
wherein, the guiding unit collects a test sample through the transporting end and provides at least a part of the test sample to react with the detection reagent.

2. The detection device according to claim 1, wherein the detection device is cooperated with a tea bag.

3. The detection device according to claim 1, wherein the wire transporting unit transports the test sample along a first direction, the guiding unit transports the test sample along a second direction, and an included angle is formed between the first direction and the second direction.

4. The detection device according to claim 3, wherein the included angle is between 20 and 90 degrees.

5. The detection device according to claim 1, further comprising:
a plurality of reaction units, wherein the carrying unit comprises a plurality of holes, and the reaction units are disposed aside the holes, respectively.

6. The detection device according to claim 5, wherein the reaction units are arranged along a straight line.

7. The detection device according to claim 5, wherein the reaction units are arranged in a polygon shape or a concentric square shape.

8. The detection device according to claim 1, wherein the reaction unit further comprises:
a plurality of reaction areas arranged in a polygon shape or a concentric square shape.

9. The detection device according to claim 1, wherein at least a part of the guiding unit is an extension structure of the transporting end, and the materials of the guiding unit and the wire transporting unit comprise cotton fiber.

10. The detection device according to claim 1, wherein the guiding unit has a wire structure, a rod structure or a ball structure.

11. The detection device according to claim 1, further comprising:
a covering unit disposed at the other side of the wire transporting unit with respect to the carrying unit, wherein the covering unit and the carrying unit form a space for accommodating the wire transporting unit.

12. The detection device according to claim 1, wherein the carrying unit has at least a non-reaction area, which is processed by a hydrophobic surface treatment.

13. The detection device according to claim 12, wherein the non-reaction area surrounds the reaction area, and the hydrophilic material is exposed for absorbing the test sample collected by the sampling end.
